(19) United States
(12) Patent Application Publication
(10) Pub. No.: US 2013/0267757 A1
(43) Pub. Date: Oct. 10, 2013

(54) ANTHRAQUINONES FOR USE AS RADIOSENSITIZERS IN CANCER TREATMENT

(71) Applicants: Moshe Schaffer, Carmiel (IL); Yuval Shaked, Binyamina (IL); Benjamin Ehrenberg, Givatayim (IL)

(72) Inventors: Moshe Schaffer, Carmiel (IL); Yuval Shaked, Binyamina (IL); Benjamin Ehrenberg, Givatayim (IL)

(73) Assignee: Sensit Science Ltd., Carmiel (IL)

(21) Appl. No.: 13/993,560

(22) PCT Filed: Dec. 15, 2011
(86) PCT No.: PCT/IL11/50060

§ 371 Date: Jun. 12, 2013

Related U.S. Application Data
(60) Provisional application No. 61/423,793, filed on Dec. 16, 2010.

(51) Int. Cl. A61N 5/10

(52) U.S. Cl. CPC A61N 5/10
USPC 600/1; 435/375

(57) ABSTRACT

Provided is an a combination therapy including a radiosensitizing agent and radiation, and being effective to inhibit proliferation of cancer stem cells. Further provided is a combination which includes a radiosensitizing agent and radiation, and effectively reduces incidence of at least one of cancer relapse and metastatic cancer in a subject having a cancer, wherein the cancer includes cancer stem cells. Also provided are anthraquinone derivatives for use as radiosensitizing agents in combination with radiations, the combination therapy being cancer specific. Further, the radiosensitizing agents were found to be cancer specific, namely, some being more effective against a cancer type as compared to others.

### FIELD OF THE INVENTION

The invention concerns radiosensitizing agents and their use in cancer treatment and prevention.

### BACKGROUND OF THE INVENTION

In recent years, new challenges in cancer therapy have been emerging. Enhanced resistance of various types of cancers to conventional treatment is observed. In addition, after the regression of the primary cancer, high incidence of cancer relapse is detected.

Enhanced cancer resistance to therapy is associated with a range of mechanisms, including mutations or over expression of the drug target, inactivation of the drug or elimination of the drug from the cells. However, even in cases where the primary cancer responds and an initial induction of cancer remission is detected, cancer may frequently relapse or reoccur after a time period.

As appreciated in the art, cancer lesions are considered to encompass a heterogeneous population of cells and comprise several types of cells. This cellular heterogeneity may be one of the causes for treatment failure, cancer reoccurrence and relapse.

Several models for cancer resistance and reoccurrence have been recently associated with small populations of cancer stem cells being identified, for example in malignancies of haematopoietic origin and some solid cancer. Such cells are also referred to as cancer initiating cells. These cells are different than most cancer cells in their unique ability to self-renewal and thus have been considered as a fundamental concept in tumor biology [1, 2].

Cancer stem cells usually express organ specific markers and have many characteristics that separate them from mature, differentiated cells. For example, interesting features of cancer stem cells is that they express high levels of specific ABC drug transporters, an active DNA repair capacity and resistance to apoptosis. In clinical terms, it is speculated that the stem cell model for reoccurrence may be highly acceptable for cancers that respond to chemotherapy with an apparent clinical complete response but relapse months or years later.

The existence of cancer stem cells may be one of the keys for the understanding to why conventional cancer therapy fails in many patients and in parallel defines a need to identify new therapeutics that can target these cells.

Currently, there are various disciplines for treating cancer. One such discipline includes a combination of radiation with the administration of a radiosensitizing agent [3, 4, 5, 6].

### SUMMARY OF THE INVENTION

The present disclosure provides, a combination therapy comprising a radiosensitizing agent and radiation therapy, and being effective to inhibit proliferation of cancer stem cells.

In accordance with a first of its aspects, the present invention provides a combination therapy comprising a radiosensitizing agent and radiation therapy, and being effective to reduce incidence of at least one of cancer relapse and metastatic cancer in a subject having a cancer, wherein the cancer comprises cancer stem cells.

In accordance with this aspect, the present invention also provides radiosensitizing agent for use in combination with radiation therapy for reducing incidence of at least one of cancer relapse and metastatic cancer in a subject having a cancer, wherein the cancer comprises cancer stem cells.

Further, in accordance with this aspect, the present invention provides a method of reducing incidence of at least one of cancer relapse and metastatic cancer comprising administrating to a patient having a cancer, with a radiosensitizing agent and subjecting the subject to radiation therapy, wherein the cancer comprises cancer stem cells.

Yet further, in accordance with this aspect, the present invention provides a kit comprising an amount of a radiosensitizing agent and instructions for use of the radiosensitizing agent in combination with radiation therapy for reducing incidence of at least one of cancer relapse and metastatic cancer in a subject having a cancer, wherein the cancer comprises cancer stem cells.

The radiosensitizing agent is preferably an anthraquinone derivative comprising the following general formula (I):

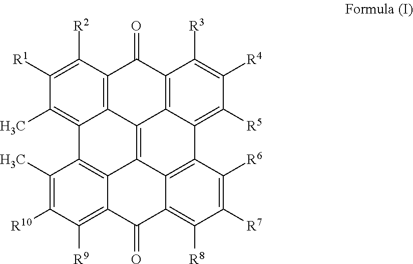

wherein

R¹ to R¹⁰ are substituents, each representing, independently, hydrogen, hydroxyl, —C₁-C₄ alkyl or —C₂-C₄ alkenyl, —C₁-C₄ alkoxy, —C₁-C₄ alkanoyl, —C₁-C₄alkanol, piperidinyl, halogen, —CX₃, —SO₃R¹¹, —R¹¹SO₂NH₂, —PO₃HR¹¹, —CO₂R¹¹, —COR¹¹; or any two adjacent substituents form together a cyclic or heterocyclic ring;

wherein

X representing a halogen; and

R¹¹ representing a hydrogen or a —C₁-C₄ alkyl;

or a salt, an ester or complex thereof, for use as a radiosentizing agent.

In some embodiments, the anthraquinone derivative of formula (I) is not hypericin.

In accordance with a further aspect (referred to at times as the cancer specific radiosensitizing aspect), the present invention provides an anthraquinone derivative of formula (I) as defined herein, for use as a cancer specific radiosensitizing agent, provided that the anthraquinone is not hypericin, or the use of the anthraquinone for the preparation of a pharmaceutical composition for radiosensitization of a specific cancer cells to radiation.

In accordance with this selective and specific radiosensitizing aspect, there is also provided a method for sensitizing a specific cancer cell (or a closed group of types of cancer) to radiation, the method comprising contacting the cancer cell with an amount of an anthraquinone derivative of general formula (I), as defined herein, the amount being effective to sensitize the cell or tissue comprising the cancer cell to the radiation therapy.

### DESCRIPTION OF THE FIGURES

In order to understand the invention and to see how it may be carried out in practice, embodiments will now be described, by way of non-limiting example only, with reference to the accompanying drawings, in which:

FIGS. 1A-1F represent chemical structures of anthraquinone derivatives, some of which being used in accordance with the present disclosure, the anthraquinone derivatives include hypericin disulfonic acid (HyDS, FIG. 1A), Hexamethyl hypericin tetrasulfonic acid (HxMeHyTS, FIG. 1B), hypericin tetrasulfonic acid (HyTS, FIG. 1C), Tetrabromo hypericin (TBrHy, FIG. 1D), and Hexamethyl hypericin (HxMeHy, FIG. 1E) as well as a non-working derivative Desmethylhypericin (DeMeHy, FIG. 1F);

FIGS. 2A-2B are schematic illustrations of the synthetic procedure for obtaining hypericin (FIG. 2A) and tetrabromo hypericin (FIG. 2B)

FIGS. 3A-3C are graphs showing fold increase (proliferation rate) of tumor cells following radiosensitizing treatment irradiation of a single dose of 10 Gy; the different figures showing the effect of treatment on C6 Rat-Glioma grown in monolayer (FIG. 3A), C6 Rat-Glioma grown as cancer stem cells spheres (FIG. 3B); and MCF7 Breast cancer grown as monolayer (FIG. 3C).

FIGS. 4A-4E are histograms showing the fold increase (proliferation rate) of various tested tumor cells treated with a Hy derivative, irradiated and evaluated 5 days post radiation; the different histograms being for C6-Rat Glioma (FIG. 4A); Dayo-human Glioma tumor (FIG. 4B); MD-MB-231-human breast carcinoma (FIG. 4C); MCF-7 human breast carcinoma (FIG. 4D) and HT29-human colon carcinoma (FIG. 4E); the different Hy derivatives being hypericin tetrasulfonic acid (HyTS), Tetrabromo hypericin (TBrHy), and Hexamethyl hypericin (HxMeHy), where Hy was used as control.

FIG. 5 is a graph showing the lack of radiosensitizing effect of Desmethylhypericin (DeMeHy) on C6 Glioblastoma tumor cells, as evaluated after treatment with 2 μM of DeMeHy and irradiation with 4 Gy on the second day of culture, the effect being compared to a control group that received no radiosensitizing agent.

FIGS. 6A-6D are graphs showing fold increase (proliferation rate) of various tested tumor cells following radiosensitizing treatment and thereafter irradiation of a single dose of 10 Gy; the different figures showing the effect of treatment on HT29-colon cancer grown in monolayer (FIG. 6A), HT29-colon cancer grown as cancer stem cells spheres (FIG. 6B); MCF-7 human breast carcinoma grown as monolayer (FIG. 6C), and MCF-7 human breast carcinoma grown as cancer stem cells spheres (FIG. 6D).

FIG. 7 is a graph showing the growth of U87 tumor cells implanted in mice following radiation and treatment with various radiosensitizing agents.

FIGS. 8A and 8B are graphs showing the growth of U87 tumor cells (FIG. 8A) and PANC01 human pancreatic adenocarcinoma cells (FIG. 8B) implanted in mice following radiation and treatment with various radiosensitizing agents.

### DETAILED DESCRIPTION OF THE INVENTION

The present disclosure is based on the finding that various radiosensitizing agents are effective in the treatment of cancer stem cells; specifically, it was found by the inventors that several anthraquinone derivatives, and in particular, hypericin derivatives, when combined with radiation, inhibit the growth of some type of cancer cells and in particular cancer stem cells.

Hypericin is a red-colored anthraquinone-derivative found naturally in the herbal remedy plant Hypericum (St. John's wort). Hypericin can be used as an antidepressant and antiviral agent, it is known to be a potent protein kinase C inhibitor and as a photosensitizer in photodynamic therapy [7]. Several studies describe the effectiveness of hypericin as a radiosensitiser [3, 4, 5, 6].

Specifically, as will be evident from the experimental results provided herein, the growth of cancer stem cells was effectively inhibited after being treated with a hypericin derivative, in combination with radiation, such as those defined by formula (I). Even more, several tested cell lines of cancer stem cells were found to be more sensitive to the combined therapy than their corresponding cancer cells (non-stem cell), suggesting that such treatment may reverse resistance of stem cell related cancers or prevent the recurrence of cancers having cancer stem cells.

Further, when mice having glioblastoma cancer were treated with a radiosensitizing agent in combination with radiation or with radiation alone, and then sacrificed, no evidence of cancer stem cells was observed in the tumors of mice treated with radiosensitizing agent in combination with radiation compared to the tumors of mice treated with radiotherapy alone, in which a substantial percentage of cancer stem cells was observed. These results were obtained from flow cytometry analysis of cancer stem cells (results not shown).

Thus, in accordance with a first of its aspects, the present disclosure provides a combination therapy comprising a radiosensitizing agent and radiation, the combination therapy being effective to inhibit proliferation of cancer stem cells.

Since cancer stem cells are believed to be associated with cancer resistance and reoccurrence, the present disclosure provided a combination therapy comprising a radiosensitizing agent and radiation, the combination therapy being effective to reduce incidence of at least one of cancer relapse and metastatic cancer in a subject having a cancer, wherein the cancer comprises cancer stem cells.

In addition, the present disclosure provides a radiosensitizing agent for use in combination with radiation for reducing incidence of at least one of cancer relapse and metastatic cancer in a subject having a cancer, wherein the cancer comprises cancer stem cells, as well as a method of reducing incidence of at least one of cancer relapse and metastatic cancer comprising administrating to a patient having a cancer, with a radiosensitizing agent and subjecting the subject to radiation, wherein the cancer comprises cancer stem cells.

As appreciated, the method according to the present disclosure may use conventional radiation protocols (e.g. type of radiation, amount of radiation, schedule of radiation etc.) or may require adaptation of a new radiation protocol, e.g. with radiation intensity lower than that provided in conventional anti-tumor radiation treatment and/or increase intervals between radiations.

In some embodiments, the patient having cancer is subjected to one or more doses of radiation after being given one or more doses of radiosensitizing agent being sufficient to enhance radiosensitization of the cancer cells to the radiation. As a result, the proliferation of the cells is inhibited and the cancer (i.e. the malignant neoplasm) is destroyed, with a reduced risk of recurring, due to the destruction also of any cancer stem cells present in the cancerous lesion.

In accordance with this aspect of the invention, there is also provides a kit comprising a composition comprising the radiosensitizing agent (one or more types of agents) and instructions for use of the radiosensitizing agent in combination with radiation therapy for reducing incidence of at least one of cancer relapse and metastatic cancer in a subject having a cancer, wherein the cancer comprises cancer stem cells.

The kit according to the present invention may be used in the method as described herein.

As used herein, the term “radiosensitizing agent” which may be read also as a “radiosensitizer” denotes an agent having an effect of enhancing the sensitivity of tumor cells to radiation.

According to some embodiments, the radiosensitizing agent is an anthraquinone derivative comprising the following general formula (I):

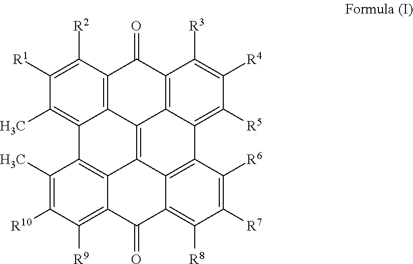

wherein
R¹ to R¹⁰ are substituents, each representing, independently, hydrogen, hydroxyl, —C₁-C₄ alkyl or —C₂-C₄ alkenyl, —C₁-C₄ alkoxy, —C₁-C₄ alkanoyl, —C₁-C₄alkanol, piperidinyl, halogen, —CX₃, SO₃R¹¹, —R¹¹SO₂NH₂, —PO₃HR¹¹, CO₂R¹¹, —COR¹¹; or any two adjacent substituents form together a cyclic or heterocyclic ring;

wherein

X representing a halogen; and

R¹¹ representing a hydrogen or a —C₁-C₄ alkyl;

or a salt, an ester or complex thereof.

In some preferred embodiments, the anthraquinone derivative of formula (I) is not hypericin.

As used herein, “C₁-C₄ alkyl” denotes a saturated, straight or branched, aliphatic chain of 1 to 4 carbon atoms, thus including, methyl, ethyl, propyl, isopropyl, butyl, isobutyl, tert butyl, sec-butyl.

As used herein, “C₂-C₄ alkeyl” denotes an unsaturated, straight or branched, aliphatic chain of 1 to 4 carbon atoms, thus including, ethenyl, propenyl, isopropenyl, butenyl, isobutenyl, tert butenyl, sec-butenyl.

As used herein, “C₁-C₄ alkanol” denotes a saturated or non-saturated, straight or branched, alcohol comprising 1 to 4 carbon atoms. This may include, without being limited thereto, any of the alcohols methyl alcohol, ethyl alcohol or tert-butyl alcohol, n-propyl alcohol, iso-propyl alcohol (sec-propyl alcohol), n-butyl alcohol, iso-butyl alcohol (sec-butyl alcohol).

Similarly, “C₁-C₄ alkoxyl”, and “C₁-C₄ alkanoyl” denote, respectively, a saturated or non-saturated, straight or branched, alkoxyl or acyl comprising 1 to 4 carbon atoms.

Further, as used herein, “halogen” or “halo” denote a halogen selected from Cl, Br, F, and I.

Yet further, when referring to “cyclic or heterocyclic ring” it is meant that two adjacent substituents, i.e. having a single or double valence bond separating therebetween form a saturated or non-saturated C₅-C₇ cyclic or heterocyclic ring, the heterocyclic ring may contain one or more elements other than carbon, the selected from sulfur, oxygen, or nitrogen.

In one embodiment, the anthraquinone derivative of formula (I) comprises identical pairs of substituents, namely pairs comprising substituents R¹ and R¹⁰; R² and R⁹: R³ and R⁸: R⁴ and R⁷: R⁵ and R⁶. In other words, at least one of the following pairing exists: R¹ is identical to R¹⁰, R² is identical to R⁹, R³ is identical to R⁸, R⁴ is identical to R⁷, and R⁵ is identical R⁶.

In some particular embodiments, R¹═R¹⁰, R²═R⁹, R³═R⁸, R⁴═R⁷, and R⁵═R⁶ (“═” indicating that the two are the same)

While the substituents R¹, to R¹⁰ may have the various meanings provided above, in one preferred embodiment, one or more of the following exist:
R¹ and R¹⁰ represent a hydrogen, halogen or an —SO₃R¹¹;
R² and R⁹ represent a hydroxyl or alkoxyl, the alkoxy group is preferably methoxy or ethoxy;
R³ and R⁸ represent a hydroxyl or alkoxyl, the alkoxy group is preferably methoxy or ethoxy;
R⁴ and R⁷ represent a hydrogen, a halogen or an —SO₃R¹¹;
R⁵ and R⁶ represent a hydroxyl or alkoxyl, the alkoxy group is preferably methoxy or ethoxy;
R¹¹ representing a hydrogen or a —C₁-C₄ alkyl.

In some preferred embodiments, R¹, to R¹⁰ has the various meanings provided below:
R¹ and R¹⁰ represent a hydrogen, bromide or —SO₃H;
R² and R⁹ represent a hydroxyl or methoxy;
R³ and R⁸ represent a hydroxyl or methoxy;
R⁴ and R⁷ represent a hydrogen, a bromide or —SO₃H;
R⁵ and R⁶ represent a hydroxyl or methoxy.

As appreciated, the term bromide denotes a —Br group and the term methoxy denotes a —OCH₃ group.

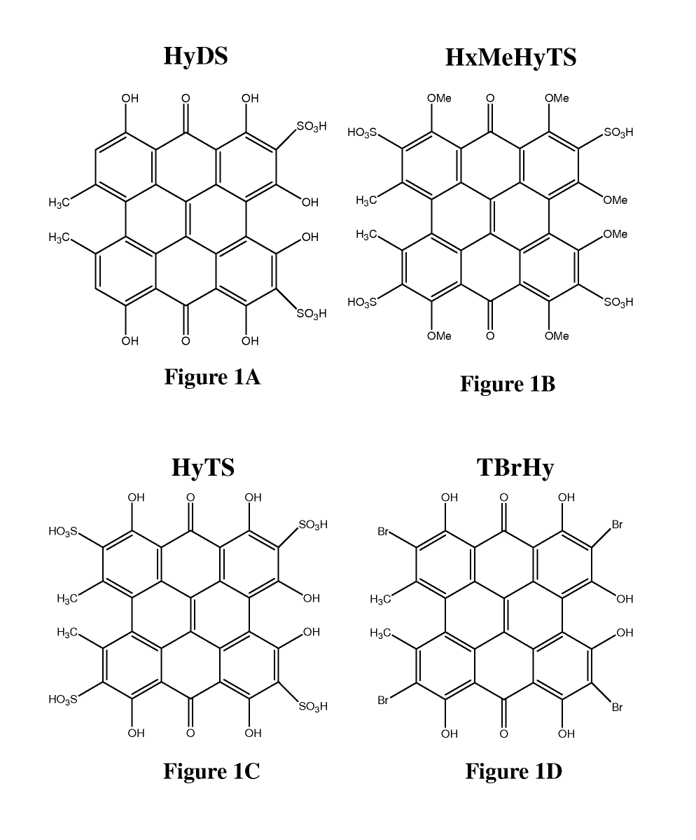

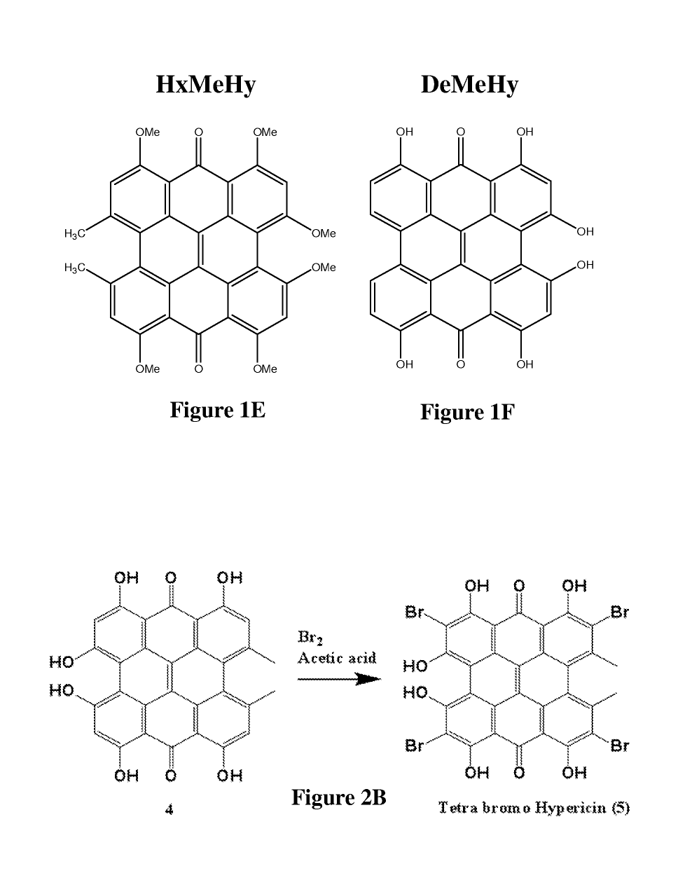

Some anthraquinone derivatives are illustrated in FIGS. 1A-1E and include the following abbreviations: hypericin disulfonic acid (HyDS), Hexamethyl hypericin tetrasulfonic acid (HxMeHyTS), hypericin tetrasulfonic acid (HyTS), Tetrabromo hypericin (TBrHy), and Hexamethyl hypericin (HxMeHy). While not shown in FIGS. 1A-1E, these derivatives were used in the form of a sodium salt.

In line with the specific anthraquinone derivatives illustrated in FIG. 1, when the anthraquinone derivative is HxMeHy, in refers to a derivative of formula (I) wherein R¹, R⁴, R⁷ and R¹⁰ are H and R², R³, R⁵, R⁶, R⁸ and R⁹ are methoxy;

When the anthraquinone derivative is HyTS it refers to a derivative of the formula (I) wherein R¹, R⁴, R⁷ and R¹⁰ are —SO₃H and R², R³, R⁵, R⁶, R⁸ and R⁹ are hydroxyl.

When the anthraquinone derivative is TBrHy is refers to a derivative having the formula (I) wherein R¹, R⁴, R⁷ and R¹⁰ are bromide and R², R³, R⁵, R⁶, R⁸ and R⁹ are hydroxyl.

The anthraquinone of formula (I) may be in a form of a salt, an ester or it may be complexed with another compound. When in the form of a salt, the counter ion may be any physiologically acceptable ion, and a non-limiting list thereof includes sodium, potassium and the like.

When in the form of an ester, any one of the substituents may be replaced with a C₁-C₄ ester. Examples of possible esters include methylester, ethylester, propylester, butylester.

The anthraquinone of formula (I) may be administered in the form of a complex. For example, the radiosensitizing agent according to the invention, may be covalently or non-covalently linked to a low molecular weight (up to 500 Da) compound, such as pyridinium or to a high molecular weight (more than 500 Da) polymer, such as a protein, polyshaccharide and the like.

In some embodiments, the radiosensitizing agent according to the invention may be a result of a combination with one or more (additional) agents selected from the group consisting of a contrasting (imaging) agent, targeting agent (moiety), drug, such as a cytotoxic drug etc. The combination may include chemical linkage between the additional agent and the anthraquinone derivative or no chemical linkage is formed therebetween and the two are used, together, albeit each in its free form. The chemical linkage may be covalent or non-covalent (e.g. electrostatic) linkage.

Non-limiting examples of contrasting agents that may be used in the context of the present disclosure include radiocontrasting agents such as Gadolinium, Mangandipyridoxyl-5′-diphosphat (Mn-DPDP), Boron-neutron capture (BNCT) as well as other types of contrasting agents, e.g. those used in x-ray imaging, such as Ultravist, Imeron, Iopathek, Isovist, Omnipaque, Visipaque, Xenetix, Dotarem, Gadovist, Magnevist, Multihance, Omniscan, Primovist, Prohance, Vasovist, Endorem, Teslascan.

The use of a targeting agent is typically aimed to increase the therapeutic index of the active agent (in this case, the derivative used as a sensitizing agent) by directing the active agent, and thus making it more available at the target cells (in this case, tumor cells). This may, inter alia, result in fewer side effects, enhancement of therapeutic efficacy, and improve subject's compliance.

The selection of a targeting agent to be used in combination with the antrhaquinone derivative will depend on the type of the tumor cell or tissue. When referring to cancer cells, non-limiting examples of targeting agents that may be used include various growth factors, kinases and others.

Similarly, when combining the anthraquinone derivative of formula (I) with a drug, the type of the drug may vary depending, inter alia, on the type of the tumor tissue and other considerations known to those versed in the art of medicine or pharmacy.

As to radiation (referred to at times by the term “radiation therapy”) it is to be understood as encompassing any as ionizing radiation known to those versed in the art or radiation. Generally, radiation therapy, and in particular ionizing radiation includes applying to the region of interest, such as a region comprising the tumor cell one single dose of ionizing radiation (“single dose”) or two or more fractions of ionizing radiation. The ionization radiation is defined as an irradiation dose which is determined according to the tumor nature and therapeutic decision. (“fractionated doses” including, for example, conventional fractionation, hyperfractionation, hypofractionation, accelerated fractionation). When referring to “irradiation dose” is it typically understood to include energies at between 2-10 Gy in each irradiation dose (session). The amount of radiation should be sufficient to damage the highly proliferating cells' genetic material, making it impossible for the irradiated cells to continue growing and dividing.

The fractionated irradiation would most likely vary from daily (e.g. several times per week) doses given for a period of weeks, or to once weekly doses given for a period of weeks. In some embodiments, the period is between 4 to 8 weeks. In some embodiments, the patient under treatment receives a radiation dosage of about 1.8-2 Gy per day for five days, repeated for several weeks, determined according to the tumor nature and therapeutic decision. The total dose and the radiation regimen will depend, inter alia, on the cancer type, type of radiosensitizing agent, irradiated area, physical condition of the patient and many other considerations appreciated by those versed in radiation therapy.

As an Example, a subject may receive a total radiation dosage of about 70 to about 80 Gy over a period of 7 to 8 weeks, each individual radiation dose to be given within approximately 1 to 24 hrs after administration of the radiosensitizing agent disclosed herein (the time being inter alia dependent on the radiosensitizing agent's pharmacokinetics).

As an additional example, a subject may receive one to three radiation sessions with a irradiation dose between 6-12 Gy for a radiation session to obtain a total dose of 18 to 24 Gy. A radiation session may include a single radiation or fractionated radiation.

As an addition example, a subject may receive irradiation for a large irradiation field (e.g. pelvis) with 1.8-2 Gy for a total dose of 45-50 Gy, adding Boost irradiation with 1.8-3 Gy (a small/shirked field, only to tumor area) per session to a total dose of between 10-20 Gy.

Such sequences of radiosensitization treatments and ionizing irradiation are repeated as needed to abate and, optimally, reduce or eliminate the spread of the cancer cells and in particular the cancer stem cells.

The radiosensitizing agent can be administered in one or more doses, at least a portion thereof being given to the patient prior to the patient's exposure to a radiation session. When the treatment schedule involves administration of several doses of the agent, the doses may be the same or different, e.g. escalating or de-escalating amounts per administration. In addition, when referring to a radiosensitizing agent it should be understood as also encompassing a combination of such agents.

In the context of the present disclosure, when referring to sensitization, it is to be understood as referring to enhancement by at least 10%, at times by at least 20%, 30%, 40%, 50%, 60%, 70% 80%, 90% and even at times by 99-100% of the inhibitory or damaging effect of the radiation on the cancer cells as compared to the effect of radiation of the same cells, without said sensitization.

As shown in the examples provided below, the combination therapy provided enhanced inhibition of the cancer cell proliferation compared to that obtained when the radiosensitizing agent or radiation or provided alone (i.e. no combination).

When referring to “inhibition of cell proliferation” is to be understood as an arrest in the proliferation rate of the cells resulting in reduction in the number of cells or elimination of the cells to a non-detectable number of cells. When referring to cancer cells, the inhibition of cell proliferation would typically result in a subsequent reduction in tumor size or total elimination of the cancerous lesion tumor

According to some particular embodiments, the combination therapy is effective in reducing incidence of cancer relapse and/or metastatic cancer. This is based on the finding that the proposed combination therapy was effective in reducing the number of cancer stem cells, which are not, or significantly less, reduced when no combination treatment is applied. For example, mice with glioblastoma were treated with a radiosensitizing agent in combination with radiation or with radiation alone. After being sacrificed, no cancer stem cells or a decreased numbers of cancer stem cells was observed in the tumors of mice treated with radiosensitizing agent in combination with radiation compared to the tumors of mice treated with radiotherapy alone. These results were obtained from flow cytometry analysis of cancer stem cells.

Cancer relapse or metastasis are well known in the art and involve the formation of a secondary cancer lesion, after some remission of the same cancer, at the initial site of formation of the primary cancer (same cell type and same location of the primary cancer) or the migration of the original cancer cells from the initial site of the cancer, to form a cancer at a different location within the body. Cancer relapse or metastasis is not limited to a time after initial treatment and may involve early relapse, for example, weeks or several months after termination of the treatment or a late relapse for example years after treatment.

In this context, a primary cancer is understood to refer to the cancer growing at the anatomical site where cancer progression began (the original site where it first arose); while a secondary cancer denotes the recurrence of a cancer, either at the site of the primary cancer or at a remote site (metastasis), As appreciated, following initial treatment of cancer either by surgical removal of most or a substantial fraction of malignant cells from a cancer patient or by treatment induced by chemotherapy, genetic therapy and the like, cancer relapse is often detected.

The process of metastasis describes cancer cells that break away from the primary cancer, leave the original cancerous site and migrate to other parts of the body via the bloodstream or the lymphatic system. For example, metastatic oral cancers usually travel through the lymph system to the lymph nodes in the neck. It is postulated by the inventors that the cells responsible for metastatic cancer or cancer relapse include cancer stem cells. Thus, the combination therapy can be applied to destroy these cancer stem cells and thus prevent the migration of cells from the primary cancer site.

When referring to reduction of incidence of relapse and/or metastasis, it is to be understood and meaning reduction of the chance of one of relapse and metastasis from occurring by a time pre-determined for a specific cancer, as its relapse time. In other words, as appreciated by those versed in oncology, various cancers can be characterized by one or more relapse times at which time points subjects treated for the cancer return to the clinic for assessing the success of treatment or verifying that there is no relapse. According to the invention, if after the combined therapy of the invention, at these time points, the cancer has not reoccurred or there is no metastatic lesion, or these time points have been significantly extended, it should be indicative of the success of the combined therapy.

The reduction of incidence of relapse and/or metastasis should be statistically significant as determined by a conventional statistical test. The reduction of incidence of relapse or metastasis will typically correlate with in vitro studies where the percent of in vitro cancer cells post treatment is compared to that of a non-treated group or any other selected control group. This correlation would be even more significant when comparing effect on cancer stem cells, taking into consideration their typical resistance to treatment. In other words, if in vitro treatment of a cancer stem cell is found to be effective, there is greater expectation that the same beneficiary and therapeutic effect would be exhibited in vivo following the combined therapy according to the invention

The cancer cells may be of any type. However, in a preferred embodiment, the cancer cells is solid cancerous cells.

When referring to “solid cancer” it is to be understood as encompassing any neoplastic mass. Thus the term excludes tumors of the blood, bone marrow and lymphatic system. Cancerous mass may show partial or total lack of structural organization and functional coordination with normal tissue and may be a primary cancerous mass or a secondary metastatic cancerous mass (i.e. as a result of cell migration from the original tumor site through the blood and lymph vessels).

Examples of solid cancers include, but are not limited to, cancers of the brain, prostate, breast, colon, lung, kidney, bladder, liver, bone, head, neck, stomach, larynx, esophagus, cervix, rectum, colorectum and other sites in the gastrointestinal tract, uterus, ovary, skin (e.g., metastatic melanomas), lymphomas (including non-Hodgkin's, Burkitt's, diffuse large cell, follicular and diffuse Hodgkin's) endometrium, pancreas and testes.

In some particular embodiments, the cancer is selected from the group consisting of breast cancer, lung cancer (e.g. small cell and non-small cell lung carcinoma), prostate cancer, colorectal cancer (including cancerous growths in the colon, rectum and appendix), brain cancer (e.g. glioblastoma), colon cancer and pancreas cancer (e.g. exocrine pancreatic cancers or adenocarcinoma). In a preferred embodiment the solid cancer comprises cancer stem cells. The existence of cancer stem cells within the cancerous lesion may be determined by detecting the presence of specific markers in histopathology analysis of biopsy removed from the cancer lesion of the subject diagnosed with cancer.

For example, CD133 is a surface marker glycoprotein also known in humans and rodents as Prominin 1 (PROM1) is expressed on many stem cells, and was found to be abundantly expressed on cancer stem cells of glioblastoma, colon carcinoma, and pancreatic adenocarcinoma.

Further, the lack of the surface marker CD24 glycoprotein expression and the expression of CD44 cell-surface glycoprotein in breast cancer cells represent population cancer stem cells.

As appreciated, cancer stem cells are cancer-forming cells that posses the ability to give rise to all cell types found in a particular cancer sample and which may generate tumors through the stem cell processes of self-renewal and differentiation into multiple cell types. Cancer stem cells are thought to be the tumor initiating cells, therefore they can initiate tumor growth and metastases.

In some particular embodiments, the cancer is selected from the group consisting of: melanomas, gliomas, breast, colon, pancreas, prostate, lung, esophagus, stomach, liver and head and neck tumors.

In some other embodiments, the solid cancer is selected from the group consisting of breast cancer, brain cancer (e.g. glioblastoma), colon cancer and pancreas cancer (e.g. exocrine pancreatic cancers or adenocarcinoma).

According to some other embodiments, the radiosensitizing agent is photofrin.

As evident from the results provided herein, the radiosensitizing agents show tumor type specificity. In other words, not only that the radiosensitizing agent is selective to tumor tissue (i.e. preference over a healthy tissue) but it also exhibits specificity to particular cancer type or types (one or more solid cancers). This is to be understood as an effect with preference to some cancers over others but does not exclude an effect of a radiosensitizing agents effect to several cancers, even if the effect over some of the agents is at lower level than known radiosensitizing agents. Thus, in accordance with a further aspect of the invention there is provided an anthraquinone derivative comprising the general formula (I), as defined herein, provided that the derivative is not hypericin, for use as a radiosentizing agent with cancer specificity.

When referring to selectivity it is to be understood that the anthraquinone derivative has greater radiosensitizing effect tumor cells (cancer cells) over a healthy tissue. When referring to specificity, it is to be understood that the anthraquinone derivative has a sensitizing effect on a specific cancer or group of cancers that is at least 10% greater, at times even 20%, 30%, 40%, or more than 50% greater than its effect on another tumor cell type. Similarly, for a derivative that is specific to a cancer, the combined therapy making use of the anthraquinone derivative and a radiation protocol would have at least 10% greater, at times even 20%, 30%, 40%, or more than 50% greater inhibitory effect on the cancer cells to which it is specific as compared to any other derivative being given with the same radiation protocol to the same type of cancer cells.

In accordance with some embodiments, the combination therapy comprises HxMeHy for use as a radiosensitizing agent with specificity to brain cancer.

In some other embodiments, the combination therapy comprises HyTS for use as a radiosensitizing agent with specificity to breast cancer, pancreatic cancer and colon cancer.

In some additional embodiments, the combination therapy comprises TBrHy for use as a radiosensitizing agent with specificity to breast cancer, pancreatic cancer, brain cancer and colon cancer.

The radiosensitizing agents according to this aspect of the invention may be used in combination with at least one additional radiosensitizing agent. For example, the anthraquinone derivative of formula (I), may be used in combination with tirapazemin or RSR-13 known to enhance the presence of oxygen in tumor tissue and thereby, without being thereto, may result in an increased radiosensitizing effect as compared to that of the anthraquinone derivative of formula (I), or tirapazemin or RSR-13 when given alone.

It is noted that the anthraquinone derivatives used in accordance with this aspect of the invention preferably exclude hypericin (Hy) or any demethylated derivatives of Hy, such as that disclosed in FIG. 1F (DeMeHy). Without being bound by theory, it appears, based on the results presented herein, that the presence of methyl substituents of the compound of formula (I) may be essential for providing the radiosensitizing activity. This hypothesis may be supported by the lack of activity of DeMeHy as shown in FIG. 5. Specifically, FIG. 5 shows that Glioblastoma cells subjected to irradiation either after treatment with DeMeHy were not sensitization of the cells to radiation by DeMeHy was exhibited. The control group included cells that received no chemical treatment before irradiation.

In some particular embodiments of this aspect, the cancer to which there is selectivity and specificity are of a type that comprises cancer stem cells.

The present invention thus provides, in accordance with another aspect, an anthraquinone derivative having the general formula (I) as defined herein, for use as a cancer (one or closed group of cancers) specific radiosensitizing agent, provided that the anthraquinone derivative of formula (I) is not hypericin. This aspect may be referred to herein at times as the cancer specific radiosensitizing aspect.

In a preferred embodiment of the cancer specific radiosensitizing aspect, the anthraquinone is specific to a cancer, such that
when said cancer is brain cancer, the anthraquinone derivative is HxMeHy;
when said cancer is breast cancer, pancreatic cancer or colon cancer the anthraquinone derivative is HyTS;
when said cancer is colon cancer, breast cancer, pancreatic cancer and brain cancer, the anthraquinone derivative is TBrHy

Also provided is the use of the anthraquinone derivative of formula I as defined herein, for the preparation of a pharmaceutical (radiosensitizing) composition for selective and specific radiosensitization of cancer cells to radiation therapy, as well as such pharmaceutical compositions. The cancer being sensitized are particularly those determined to comprise cancer stem cells.

In some preferred embodiments, the pharmaceutical composition is prepared to comprise an amount of HxMeHy, the amount being effective to sensitize brain cancer cells to radiation; or the pharmaceutical composition is prepared to comprise an amount of HyTS, the amount being effective to sensitize one or more of a breast cancer cells, pancreatic cancer cells and colon cancer cells to radiation; further or the pharmaceutical composition is prepared to comprise an amount of TBrHy, the amount being effective to sensitize colon cancer cells, breast cancer cells, pancreatic cancer cells and brain cancer cells to radiation.

The pharmaceutical composition may include, in addition to the active ingredient a physiologically acceptable carrier. The selection of the carrier will depend, inter alia, on the selected route of administration of the composition.

In one embodiment, the composition is to be administered by injection, e.g. intravenous or intraperitoneal injection.

The active ingredient may be included in the composition in a free form or in a lipid vesicle, such as liposomes. In one embodiment, the antrhaquinone derivative is encapsulated in the intraliposomal core of liposomes and is released from the liposomes either before or once at tumor cell or tumor tissue. Those versed in liposome technology will know how to select the appropriate vesicles for delivery of the antrhaquinone derivatives according to the present disclosure.

The antrhaquinone derivative may be combined with one or more additional active agents, being in the same or different pharmaceutical compositions. Further, the combination includes simultaneous as well as sequential administrations. In some embodiments, the antrhaquinone derivative is combined with an additional radiosensitizing agent. Examples of additional radiosensitizing agents were mentioned hereinbefore.

Also provided in accordance with the specific radiosensitizing aspect of the invention a method for selective and specific sensitizing cancer cells to radiation, the method comprising contacting cancer cells cancer cells with an amount of an anthraquinone derivative of general formula (I), the amount being effective to selectively and specifically sensitize the cancer cells to radiation. Particular embodiment of this method include:
when said cancer is brain cancer, the anthraquinone derivative is HxMeHy;
when said cancer is breast cancer, pancreatic cancer or colon cancer the anthraquinone derivative is HyTS;
when said cancer is colon cancer, breast cancer, pancreatic cancer and brain cancer, the anthraquinone derivative is TBrHy.

As used herein, the forms “a”, “an” and “the” include singular as well as plural references unless the context clearly dictates otherwise. For example, the term “cancer” includes one or more cancer cells.

Further, as used herein, the term “comprising” is intended to mean that, for example, the radiosensitizing composition include the recited anthraquinone derivative of formula (I), but not excluding other elements, such as physiologically acceptable carriers and excipients as well as other active agents. The term “consisting essentially of” is used to define, for example, compositions which include the recited elements but exclude other elements that may have an essential significance on treatment of tumors. “Consisting of” shall thus mean excluding more than trace amounts of other elements. Embodiments defined by each of these transition terms are within the scope of this disclosure.

Further, all numerical values, e.g. when referring the amounts or ranges of the elements forming part of the present disclosure are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore to be understood that within the scope of the appended claims, the invention may be practiced otherwise, in a myriad of possible ways, than as specifically described hereinbelow.

### DETAILED DESCRIPTION OF SOME NON-LIMITING EMBODIMENTS

### Example 1

### Synthesis of Hypericin Derivatives

The general procedure for the synthesis of hypericin and hypericin derivatives has been described [8, 9, 10]

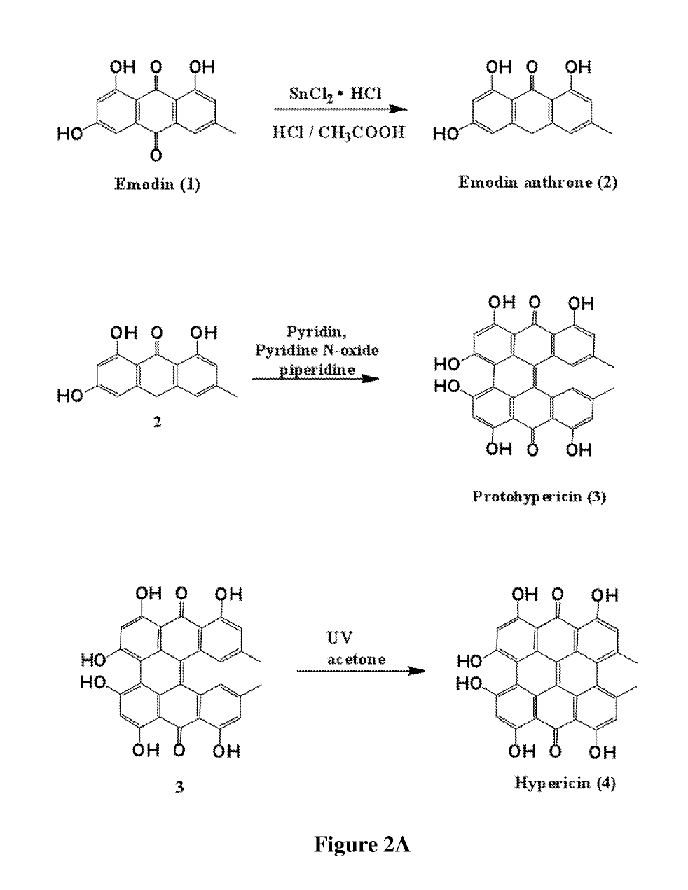

The synthetic scheme for the preparation of hypericin is provided in FIG. 2A and includes:

Emodin anthrone (2): A warm solution of tin (II) chloride hydrate (41.41 gr, 0.185 mol) in conc. hydrochloric acid (213 mL) was added to a suspension of 1 (5 g, 0.0185 mol) in acetic acid (368 mL) to give the red-colour solution. The solution was refluxed for 24 hours and it was then poured into ice-water. The precipitate was collected by filtration to yield emodin anthrone (3.76 g, 80%).

¹H-NMR (DMSO-d₆, 200 MHZ δ) of compound 2: 2.32 (s, 3H, Me), 4.31 (s, 2H, CH₂), 6.22 (d, 1H, J=2.35, H-4), 6.42 (d, 1H, J=2.35, H-2), 6.68 (s, 1H, H-5), 6.78 (s, 1H, H-7), 10.83 (s, 1H, OH), 12.22 (s, 1H, OH), 12.38 (s, 1H, OH) ppm.

Protohypericin (3): Compound 2 (1 gr, 3.9 mmol), FeSO₄.7H₂O (54 mg, 0.195 mmol), pyridine N-oxide (1.85 gr, 19.5 mmol) in pyridine (21 mL) and piperidine (1.92 mL) were placed in a 100 mL round bottom flask. The solution was then heated to 120° C. for 1.5 hr under Ar atmosphere. After cooling to room temperature the solution was poured to HCl (2N) and stirred for 30 min. The precipitate was centrifuged, washed x3 with 3% HCl and dried under high vacuum with P₂O₅. The crude product was purified by column chromatography using CHCl₃:

MeOH (8:2) as eluent to yield protohypericin.

¹H-NMR (acetone-d₆, 200 MHZ, δ) of Compound 3: 2.08 (s, 2 Me), 6.41 (s, ar-H-2+ar-H-5), 6.7 (s, ar-H-11+ar-H-12), 7.26 (s, ar-H-9+ar-H-14), 12.9, 14.33 (2s, OH-6+OH-1, OH-15+OH-8) ppm. (MS: 505 gr/mol, MH⁻)

Hypericin (4): Compound 3 was dissolved in acetone and irradiated with a 150 W high-pressure mercury lamp at room temp for 15 min. The dark red solution was dried and purified by column chromatography using CHCl₃:MeOH (8:2) as eluent to yield hypericin.

¹H-NMR (DMSO-d₆, 200 MHZ, δ) of Compound 4: 2.75 (s, 2Me), 6.60 (s, ar-H-2+ar-H-5), 7.46 (s, ar-H-9+ar-H-12), 14.08 (s, OH-8+OH-13), 14.74 (s, OH-1+OH-6) ppm. MS: (504 gr/mol, MH⁻)

The synthetic scheme for the preparation of Tetrabromo Hypericin is illustrated in FIG. 2B and includes:

Tetrabromo Hypericin (5): Compound 4 (20 mg, 39.65 μmol) was dissolved in 5 mL of acetic acid in 50 mL round bottom flask equipped with reflux condenser. A freshly solution of Br₂ (1.757 gr, 11 mmol) in 25 mL acetic acid was added and the suspension was stirred at a room temp for 2 hr. The reaction was monitored by TLC (EtOAc:MeOH; 98:2). After completion of the reaction, the solvent was evaporated to dryness and the crude product was purified by column chromatography to yield tetrabromo-hypericin (23.6 mg 72%) in the form of dark brown solid. MS: (819 gr/mol, MH⁻).

¹H-NMR (acetone-d₆, 200 MHZ, δ) of Compound 5: 2.85 (s, 6H, CH₃-10, 11), 14.9 (s, OH-8+OH-13), 15.4 (s, OH-1+OH-6) ppm.

For preparation of HyTS: 50 mg of hypericin and 250 mg oleum were kept at 75° C. for 1.5 h. The reaction mixture was gently diluted with ice water and saturated with NaCl. After centrifugation, the precipitated green precipitate was washed with cold water and thoroughly dried in vacuum. The material in question was isolated by column chromatography on Sephadex-LH20 with methanol/water (4/1) as the eluent.

For preparation of HxMeHy: Refluxing 10 mg hypericin in the presence of 0.2 ml dimethyl sulfate and 0.5 g K₂CO₃ in 6 ml acetone for 24 h. The crude product was purified by silica column chromatography using ethylacetate/water 100/2.5 with increasing amounts of acetone as eluent. The compound was eluted from the column with acetone/methanol 90/10 and was further purified by Sephadex LH-20 column chromatography as indicated for hypericin. In all cases, as a final purification step, the compounds were dissolved in a mixture of methanol and acetone and triturated with a tenfold volume of petroleum ether to precipitate them.

The other derivatives exemplified herein may be prepared in similar manner as described in the above provided references, the content of which is incorporated herein in their entirety, by reference.

### Example 2

### In Vitro Assay

### Example 2A

### Methods:

### Cell Lines, Tumor Stem Cell, and Cell Culture

The following cell lines were originally obtained from the American Type Culture collection (ATTC):

Human breast carcinoma: MCF-7 (ATCC number: HTB-22); MDA-MB-231 (ATCC number: HTB-26)

Human glioma cell lines: Daoy (ATCC number: HTB-186) and

Rat glioma cell line: C6 (ATCC number: CCL107)

Human colorectal cancer cell line: HT-29 (ATCC number: HTB-38). All cells were cultured routinely in DMEM supplemented with 10% FCS.

To obtain tumor stem cells, cells were cultured in stem cell conditioned media. Briefly, breast and glioma cancer cells were cultured in serum-free DMEM media containing 10 n/ml bovine insulin, 100 μg/ml human transferrin, 100 μg/ml BSA, 60 ng/ml progesterone, 16 μg/ml putrescine, 40 μg/ml sodium selenite, 63 μg/ml N-acetylcysteine, 5 μM forskolin, 50 units/ml penicillin, and 50 μg/ml streptomycin, 10 ng/ml bFGF, and 10 ng/ml PDGF. In all experiments, cells were maintained in 100 mm or 96 well plate culture dishes at 37° C. in a humidified 5% and were protected from light.

### Hypericin Derivatives and Doses

Three hypericin derivatives were used in the assay, including hypericin tetrasulfonic acid (HyTS); Tetrabromo hypericin (TBrHy); and Hexamethyl hypericin (HxMeHy). In addition, as control, hypericin and/or Photofrin II as well as cells exposed only to radiation, i.e. without a radiosensitizing agent.

The hypericin, hypericin derivatives and photofrin II were used in doses ranging from 0.1 μM up to 2 μM. All compounds were dissolved in DMSO, and were protected from light.

### Radiation

In all cases, control and tumor cells were exposed to ionizing radiation (Siemens X-ray device with 250 kV or with LINAC) at 24 h after the incubation with Hyperecin, Photofrin II or Hypericin derivatives indicated below. The total delivered radiation dose was a 10 Gy, provided in a single irradiation session on the second day of culture. The response of the tumor to the radiation treatment was evaluated by determining growth delay using the described cell proliferation assay.

Cell proliferation assay using AlamarBlue

Unless indicated otherwise, the tumor cell lines were cultured at a starting density of 1×10⁵ cells/mL in DMEM and 10% FCS. Cells were allowed to grow over a period of 5 days using the method of AlamarBlue™ reduction assay. In brief, 10% AlamarBlue was added, according to manufacturer's instructions, to cultured cells at the time of seeding. The AlamarBlue method allows evaluation of proliferation based on mitochondrial activity product (a standard method to assess cell proliferation). Absorbance was measured at 570 nm and 600 nm using a micro-titer plate reader. Results were plotted as percentage of reduction representing cell proliferation as further discussed below.

### Results:

Hypericin and its Derivatives can Affect Both Tumor Cells and Tumor Stem Cells when Exposed to a Single Irradiation Followed by Radiosensitization

In order to evaluate the radiosensitive properties of hypericin derivatives on cancer cells and cancer stem cells, the minimal effective concentration of the following hypericin derivatives were used (based on the litreture): Hy, TBrHy, and HxMeHy in a dose of 0.1 μM; HyTS in a dose of 1 μM and Photofrin II in a dose of 1 μg (which may be regarded as an equivalent of 0.88 μM).

The efficacy of Hypericin derivatives was tested in radiation administered at a dose of 10 Gy on breast, colon and glioma tumors. The cells were first seeded with 10% AlamarBlue solution on day 1 along with tested compounds. On day 2 cells were irradiated with 10 Gy. The results indicate that for the tested tumor cell lines, day 6 of the assay reached a saturation point. Therefore, experiments were performed for 5 days, at which point cell proliferation was documented.

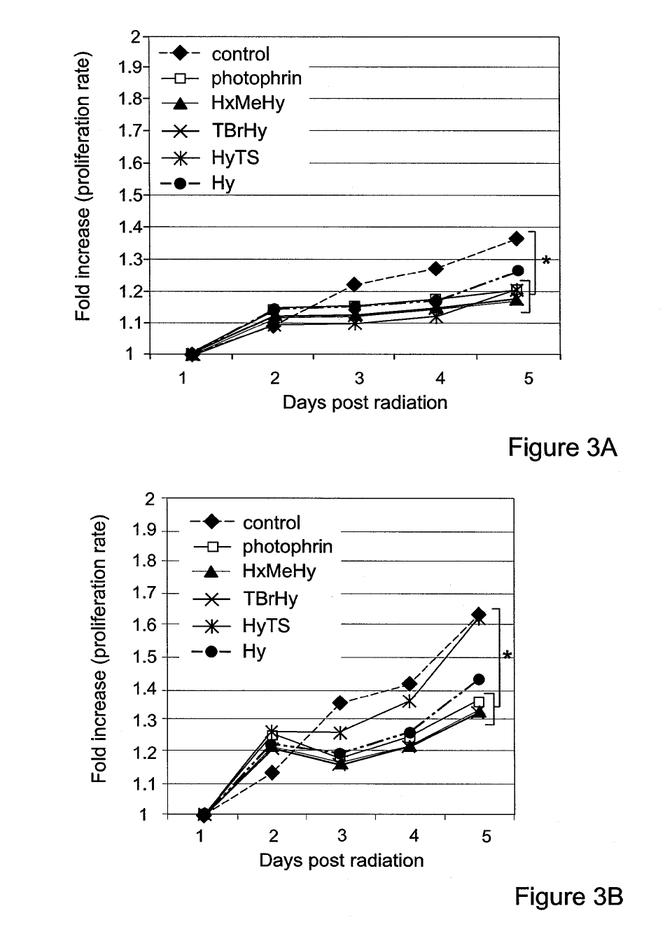

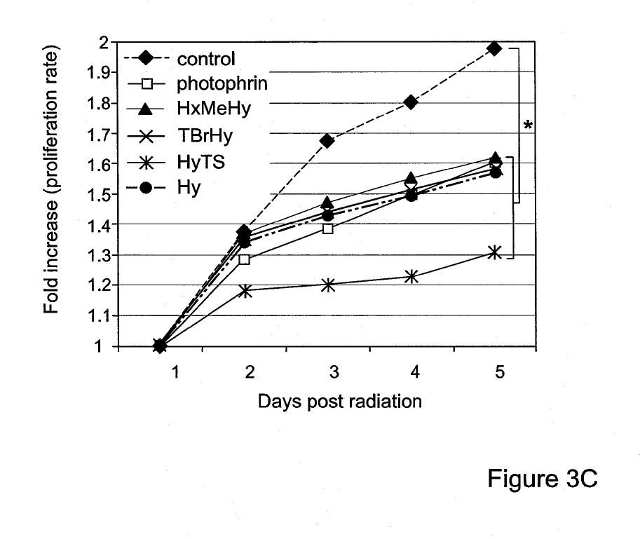

Tumor cell proliferation was assessed by AlamarBlue % of color reduction, and results were plotted as fold increase from baseline. The results in FIGS. 3A-3C show that the anthraquinone derivatives of formula (I) tested in this experiment were all effective in radiosensitizing the tested C-6 glioma cancer and breast cancer.

The results of the proliferation assay depict specificity of some hypericin derivatives in inhibiting the proliferation of certain tumors. HxMeHy at a concentration of 0.1 μM statistically inhibited the proliferation of C6 glioma cells in both monolayer and when grown as CSCs measured 5 says post irradiation of a dose of 10 Gy (FIG. 3A (P<0.05) and FIG. 3B (P<0.049), respectively, while HyTS at a concentration of 1 μM statistically inhibited the proliferation of MCF-7 breast cancer cells (grown as monolayer) measured 5 says post radiation, using a irradiation of a dose of 10 Gy (FIG. 3C (P<0.03)

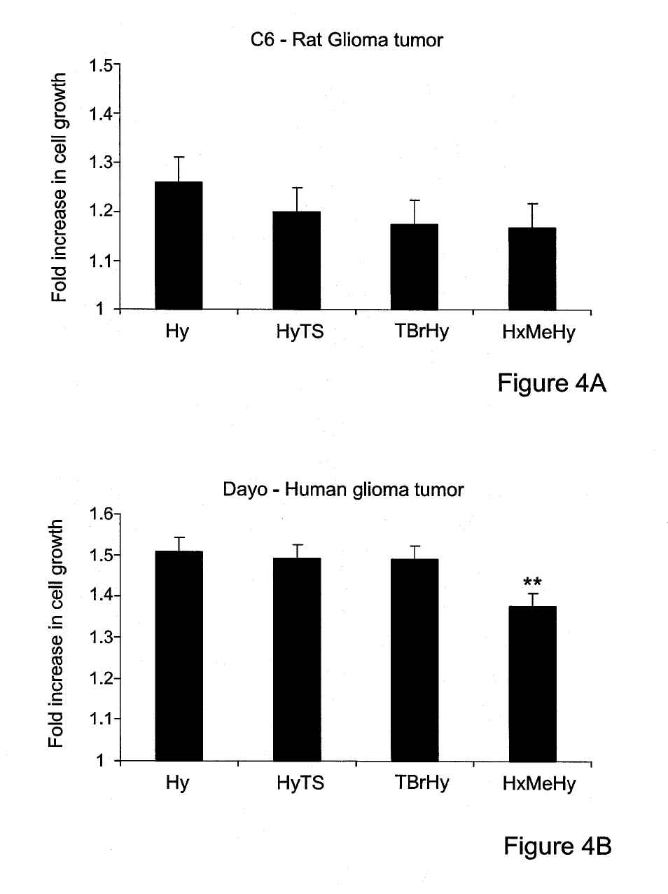

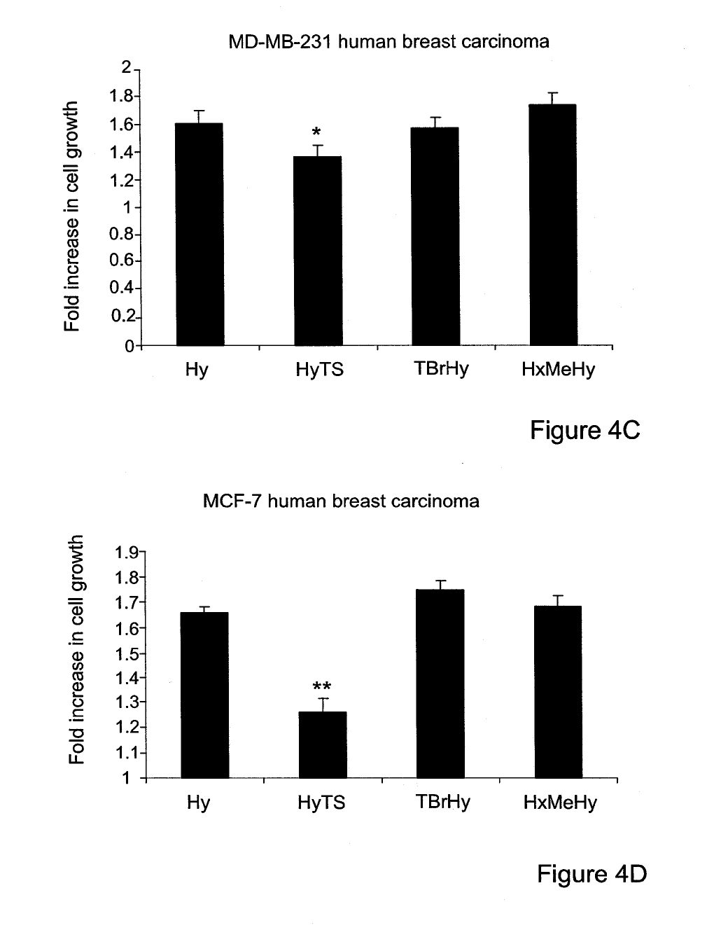

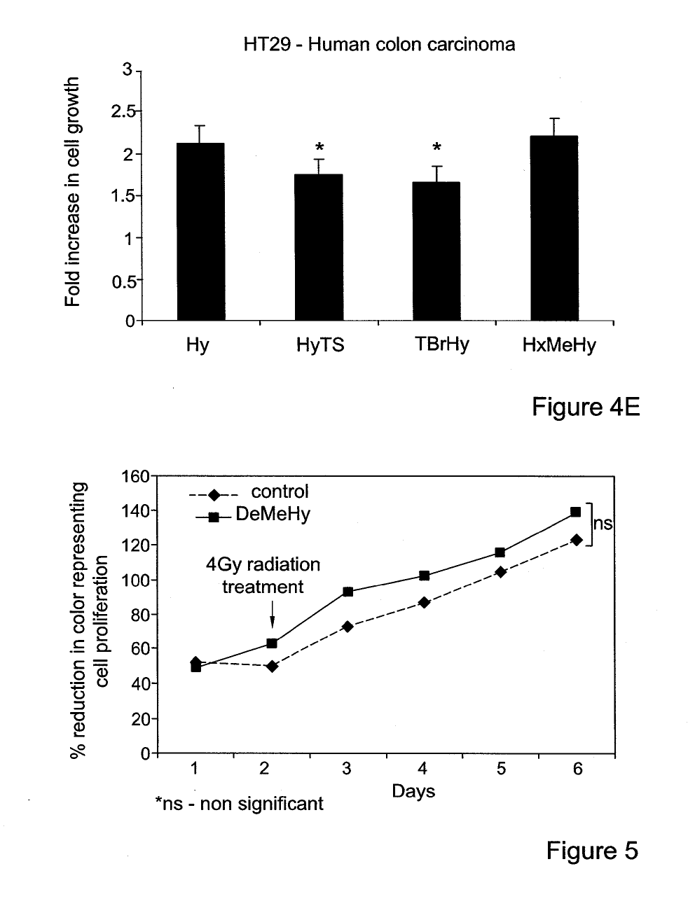

The results in FIGS. 4A-4E also show the AlamarBlue cell proliferation assay as described hereinabove. The cells were treated with the radiosensitizing agents on day “0” and irradiated on day “1” and proliferation was measured on day 5. The results showed that HxMeHy when used at a concentration of 0.1 μM is an effective radiosensitizing agent for C6 brain cancer (FIG. 4A) and Dayo tumor (FIG. 4B, P<0.01).

HyTS at a concentration of 1 μM was shown to be statistically significantly effective as a radiosensitizing agent for breast cancer (FIG. 4C-4D, P<0.05 and P<0.01 respectively). Both TBrHy at a concentration of 0.1 μM and HyTS were shown to be effective radiosensitizing agents for colon cancer (FIG. 4E, P<0.05).

FIG. 5 shows lack of radiosensitizing effect of DeMeHy on C6 Glioblastoma tumor cells, as evaluated after treatment with 2 μM of DeMeHy and irradiation with 4 Gy on the second day of culture, the effect being compared to a control group that received no radiosensitizing agent.

Overall, the results clearly indicate that hypericin derivatives act as radiosensitizing agents in various tumors, and that these agents can also reduce the proliferative properties of tumor stem cells.

### Example 2B

### Methods:

### Cell Lines, Tumor Stem Cell, and Cell Culture

Linear cell (Lin) HT29 and MCF7 human breast carcinoma, were obtained from the American Type Culture Collection (ATCC).

Cells were cultured in Dulbecco's Modified Eagle Medium (DMEM), supplemented with 10% fetal calf serum (FCS) and antibiotics.

Enrichment of the cancer stem cells (CSCs) population was done by incubating linear cells in a DMEM/F-12 (HAM) 1:1 medium supplement with Insulin-Transferin-Soldium Selenite, and growth factors specific for each cell line. Cells considered enriched for CSCs after 10-15 days in CSCs medium.

### Hypericin Derivatives and Doses

Three photosensitizing agents were used, Hypericin (Hy), Tetrabromo hypericin (TBrHy), and hypericin tetrasulfonic acid (HyTS) in the following concentrations: 0.1 μM, 1 μM and 2 μM. Drugs were protected from light for all time. Control cells were treated with 0.1% DMSO (vehicle).

### Radiation

Linear and CSC were exposed to 3 Gy irradiation daily for four consecutive days or to a single dose of 10 Gy radiation. Radiation was initiated one day after treatment with the hypericin or hypericin derivatives.

### Evaluation of Cells Proliferation

Linear and CSCs were cultured in a 96 plate in triplicates. Each cell line was treated with the three radiosensitizing agents in the indicated doses. In addition culture cells were exposure to radiation. A day before the irradiation, the radiosensitizing agents were added to the culture. Cells were allowed to grow over a period of 5 days using the method of AlamarBlue™ reduction assay. 10% Alamar Blue solution added to each well. Cells viability was measured using Eliza Reader at two different wavelengths: 570 nm and 600 nm, and the appropriate equation was calculated to reflect cell viability.

### Results:

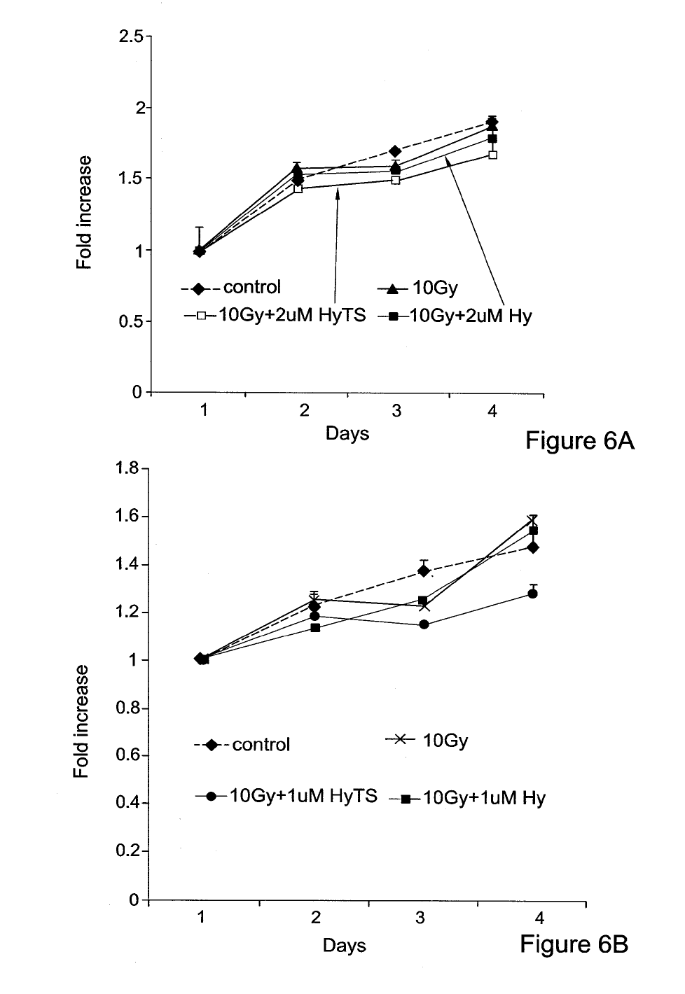

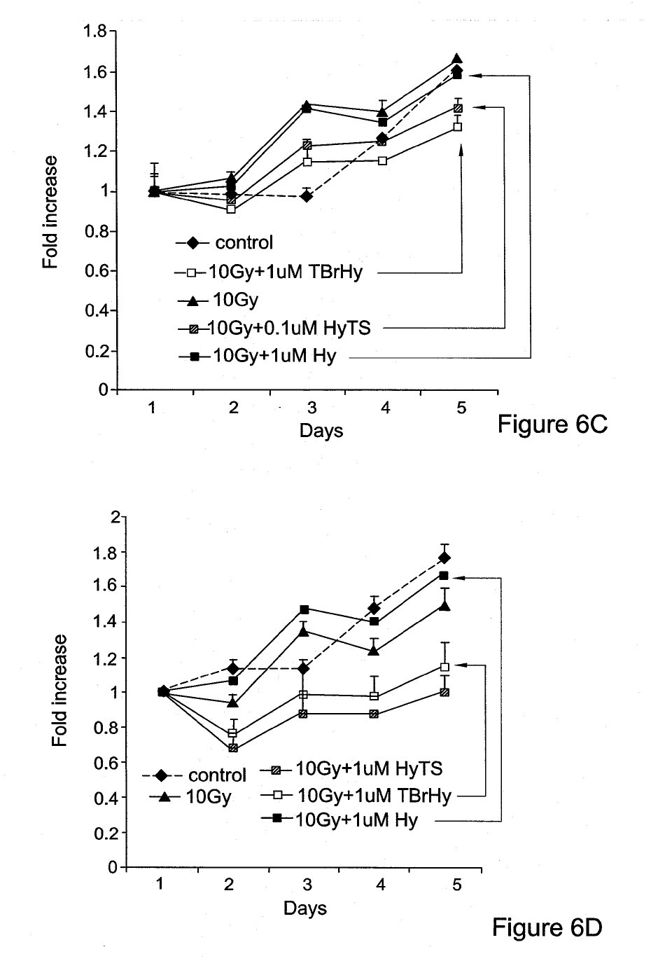

The results presented in FIGS. 6A and 6B, show that HyTS is effective in radiosensitizing HT-29 colon cancer cells. In addition, the HT-29 colon cancer stem cells are more sensitive to HyTS treatment (FIG. 6B).

FIGS. 6C and 6D show that that HyTS and TBrHy are effective in inhibiting the proliferation of MCF7 human breast cancer cells grown as monolayer and as stem cells. Noteworthy, the effect is more pronounced in the MCF7 cancer stem cells.

### Example 3

### Brain Blood Barrier (BBB) Penetration of TBRHY and HYTS

### Methods:

Brain tumor sections were obtained after harvesting the brains of mice treated with hypericin derivatives, and observed under fluorescent microscope. Hypericin derivatives were observed via fluorescent microscopy in the wavelength of 550-650 nm

### Results:

The results from the brain sections indicated that TBrHy penetrated the BBB whereas HyTS does not penetrate the BBB.

### Example 4

### In Vivo Assay

### Example 4A

### Efficacy and Toxicity of Radiosensitizing Agents

### Methods:

U87 human glioma cells at a concentration of 5×10⁶ were implanted subcutaneously into the flank of eight to ten weeks old nude mice (n=4-5 mice/group). Treatment was initiated when the tumors reached a size of 30-50 mm³ and included injection of 10 mg/Kg of one of the radiosensitizing agents Photofrin, HyTS or TBrHy. Mice injected with 5% DMSO vehicle served as the control untreated group. Immediately after the injections, the mice were placed in the dark. For each group of radiosensitizing agent treated mice, half of the treated mice were then treated with a 10 Gy dose of radiation, initiated two hours after the administration of the radiosensitizing agents. Mice were kept in the dark for the following 72 hours, in order to minimize the effect of light on the traces of the radiosensitizing agents.

Overall, eight groups of treatment were evaluated as follows:

1. Control vehicle (5% DMSO)

2. 10 Gy radiation

3. 10 mg/kg Photofrin

4. 10 mg/kg HyTS

5. 10 mg/kg TBrHy

6. 10 mg/kg Photofrin+10 Gy radiation

7. 10 mg/kg HyTS+10 Gy radiation

8. 10 mg/kg TBrHy+10 Gy radiation

Tumor growth was evaluated by measuring the tumor's volume twice weekly.

Treatment toxicity was evaluated using two parameters, (i) measuring mice's body weight and (ii) monitoring overall mice behaviour, both parameters were measured twice weekly.

### Results:

This study was conducted in order to evaluate in vivo the anti-tumor activity of radiosensitizing agents administered alone and/or in combination with radiation in U87 human glioma tumors implanted in nude mice. In addition, the potential toxicity of the various treatments was assessed.

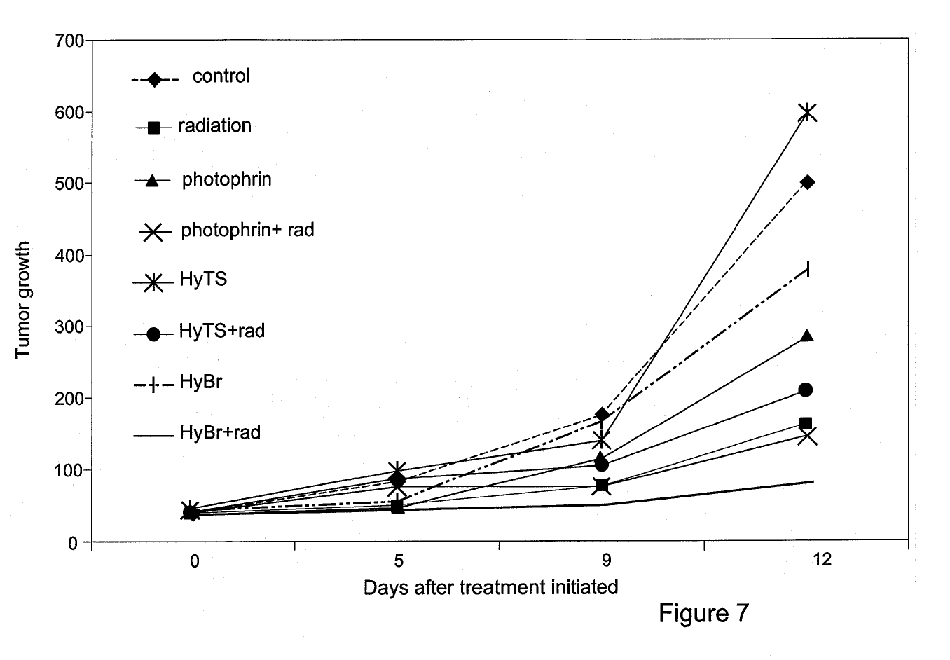

The results presented in FIG. 7 indicate that TBrHy is the most effective radiosensitizing agent, showing the maximum reduction in tumor growth when combined with radiotherapy.

The results presented herein also indicate that treatment of TBrHy in combination with radiation exhibit a better anti-tumor activity as compared to the radiation treatment. In one mouse treated with combination of TBrHy and radiation, the tumor was completely removed 4 days after treatment.

Treatment with the radiosensitizers agents: HyTS or HyBr was well tolerated as indicated by the fact that no significant change was observed in the mice body weight and in the mice behaviour. Significant change in the body weight was observed in mice treated with radiation. These results clearly suggest that treatment with HyTS or HyBr are well tolerated even when administered in combination of in high doses such as 10 mg/kg (FIG. 7).

### Example 4B

### Efficacy of Radiosensitizing Agents on U87 Human Glioma Tumors

### Methods

U87 human glioma cells at a concentration of 5×10⁶ were implanted into the flank of eight to ten weeks old CB.17 SCID mice (n=4-5 mice per group).

Treatment was initiated when the tumors reached a size of 100-150 mm³, and included the following treatment groups:

1. Control (PBS with 0.1% DMSO).

2. 10 mg/kg Hypericin+10 Gy radiation

3. 10 mg/kg HyTS+10 Gy radiation

4. 10 mg/kg TBrHy+10 Gy radiation

Specifically, mice were injected with the radiosensitizing agent, and immediately placed in the dark. Radiation was initiated two hours after radiosensitizing administration. Mice were kept in the dark for the following 72 hours, in order to minimize the effect of light on the traces of the radiosensitizing agents. Tumor volume was monitored prior to treatment and thereafter twice weekly.

### Example 4C

### Efficacy of Radiosensitizing Agents on PANC01 Human Pancreatic Adenocarcinoma CELLS

### Methods

PANC01 human pancreatic adenocarcinoma cells at a concentration of 5×10⁶ were implanted into the flank of eight to ten weeks old CB.17 SCID (n=4-5 mice per group).

Treatment was initiated when the tumors reached a size of 100-150 mm³ and included the following treatment groups:

1. Control

2. 10 Gy radiation

3. 10 mg/kg Hypericin+10 Gy radiation

4. 10 mg/kg HyTS+10 Gy radiation

5. 10 mg/kg TBrHy+10 Gy radiation

Mice were injected with the radiosensitizing agent, and immediately after were placed in the dark. Radiation was conducted 2 hours after radiosensitizing administration. Mice were kept in the dark for the following 72 hours, in order to minimize the effect of light on the traces of the radiosensitizing agents. Tumor volume was measured prior to the treatment and thereafter twice weekly.

Results:

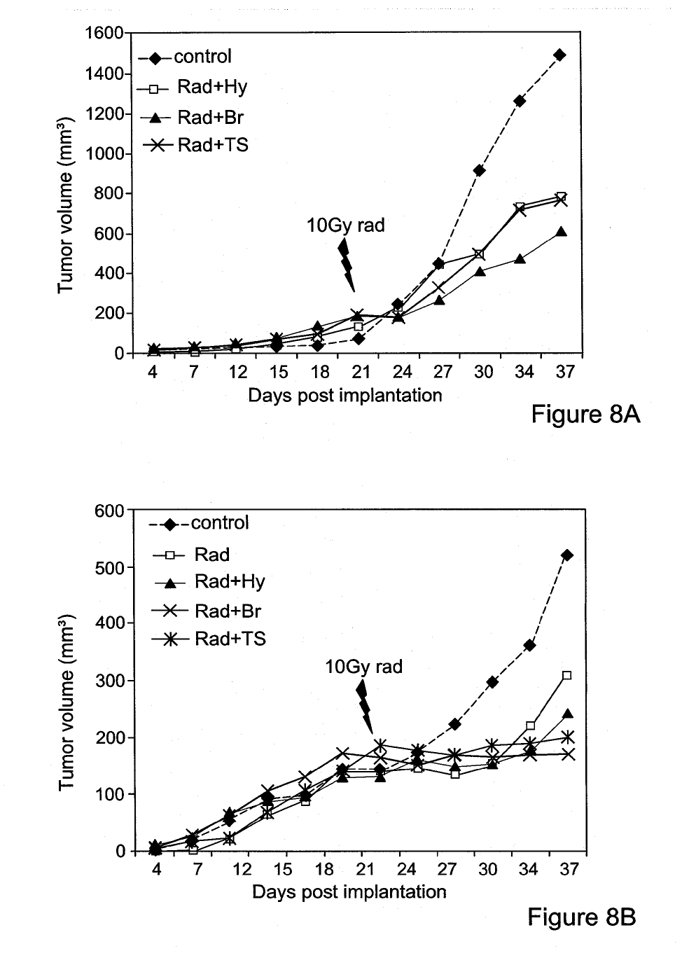

As shown in FIGS. 8A and 8B, administration of the hypericin derivatives with the radiation protocol significantly reduced the tumor size of both U87 and PANC1 tumors compared to control.

In the glioblastoma U87 tumors and in the PANC1 pancreatic tumors, TBrHy was found to be the most potent radiosensitizing agent, as shown from the maximum reduction in tumor volume obtained in the mice treated with TBrHy and radiation.

Based on results shown in FIGS. 8A and 8B, treatmetn of TBrHy in combination with radiation exhibited enhanced anti-tumor activity.

In addition, HyTS when combined with radiation, was also shown to have anti-tumor activity in both tumor models, with a somewhat better activity in PANC1 pancreatic tumors.

It should be noted that the tested tumors, namely glioblastoma and pancreatic are often very aggressive and hard to treat. Furthermore, these tumors are usually resistant to many treatment modalities of cancer including radiation therapy.

### Example 4D

### Localization of Radiosensitizing Agents in the Tumors Methods

### Animal Studies:

U87 Tumors were implanted subcutaneously in nude mice and then examined under a UV light lamp and the colour intensity was visualized.

### Single Cell Suspension Preparation:

U87 Tumors were resected when tumors reached end-point (1500 mm³), minced, and digested in 4 mg/ml collagenase III, 2 mg/ml hyaluronidase, and 2 mg/ml collagenase IV in serum free DMEM, for 20 min, at 37° C. Cells were then filtered through a 40 μm sieve, and washed twice with PBS to obtain single U87 cell suspension.

The single U87 tumor cell suspensions were then acquired on Cyan ADP flow cytometer (Beckman Coulter) and analyzed with Summit (Beckman Coulter) software.

The FL8 channel, (wavelength of 655/20 nm) was used to detect differences between positive and negative cells. From each sample, at least 50,000 cells were acquired.

### Results:

These studies were aimed at assessing the localization of radiosensitizing agents and were conducted by two complementary techniques; the first in implanted U87 human glioma tumors using UV lamp and the second in extracted U87 tumors using Flow Cytometry techniques.

U87 Tumors treated with HyTS or TBrHy were colored in red when examined under a UV light during the 2 hours prior to radiation.

In addition, in tumors treated with HyTS or TBrHy (with or without radiation) a fluorescent signal was detected at a wavelength of 665 nm by Flow Cytometry techniques.

Taken together the above results, it is postulated that there is an uptake of the radiosensitizing agent by the tumors cells and that the agent is localized in the tumor cells during the time of the experiment

### REFERENCES

1. Dean M, Fojo T, Bates S, Nature Reviews, 2005; 5: 275-285.
2. Zhou B B S, Zhang H, Damelin M, Geles K G, Grindley J C, Dirks P B, Nat Rev Drug Discov. 2009; 8: 806-23.
3. Rose H, Wagner H. in: Radiobiol. Radiother (Berl) 1965; 6: 477-83
4. Zhang W, Anker L, Law R E, Hinton D R, Gopalakrihna R, Pu Q, Gundimeda U, Weiss M H, Couldwell W T, Clin Cancer res 1996; 2: 843-6.;
5. Sun Y, Xu H, Tang S, Meng Y, Dong Z, Yang Z, Chuang L, Er Bi Yan Hou Ke Za Zhi 2005; 19: 1127-30
6. Wessels J T, Busse A C, Rave-Frank M, Zanker S, Hermann R, Grabbe E, Muller G A, Photochem. Photobiol. 2008; 84: 228-235
7. Delaey E, Zupko I, Chen B, Derycke, A, van Laff F, De Vos D, De Witte P, Int. J. Oncol. 2003; 23: 519-524
8. Cameron D. W. et al Aust. J. Chem. 1976; 29, 1521-1533
9. Brockmann H. and Lackner H. Tetrahedron Lett. 1979; 18, 1575-1578
10. Falk H. and Schmitzberger W. On the bromination of hypericin—the gymnochrome chromophores. Monatsh. 1993; 124, 77-81

1.-48. (canceled)

49. A method of reducing incidence of at least one of cancer relapse and metastatic cancer comprising administrating to a patient having a cancer, with a radiosensitizing agent and subjecting the subject to radiation therapy, wherein the cancer comprises cancer stem cells.

50. The method of claim 49, wherein the patient is subjected to radiation following administration of the radiosensitizing agent.

51. The method of claim 49, comprising one or more administrations of said radiosensitizing agent and one or more radiations.

52. The method of claim 49, wherein the radiosensitizing agent is an anthraquinone derivative having Formula (I) 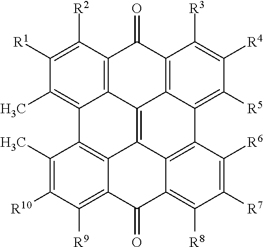
wherein
R¹ to R¹⁰ are substituents, each representing, independently, hydrogen, hydroxyl, —C₁-C₄ alkyl or —C₂-C₄ alkenyl, —C₁-C₄ alkoxy, —C₁-C₄ alkanoyl, —C₁-C₄alkanol, piperidinyl, halogen, —CX₃, —SO₃R¹¹, —R¹¹SO₂NH₂, —PO₃HR¹¹, —CO₂R¹¹, —COR¹¹; or any two adjacent substituents form together a cyclic or heterocyclic ring;
wherein
X representing a halogen; and
R¹¹ representing a hydrogen or a —C₁-C₄ alkyl;
or a salt, an ester or complex thereof.

53. The method of claim 52, wherein pairs of substituents are identical, the pairs comprising substituents R¹ and R¹⁰, R² and R⁹, R³ and R⁸, R⁴ and R⁷, and R⁵ and R⁶.

54. The method therapy of claim 53, wherein
R¹ and R¹⁰ represent a hydrogen, halogen or an —SO₃R¹¹;
R² and R⁹ represent a hydroxyl or an alkoxyl;
R³ and R⁸ represent a hydroxyl or an alkoxyl;
R⁴ and R⁷ represent a hydrogen, a halogen or an —SO₃R¹¹;
R⁵ and R⁶ represent a hydroxyl or an alkoxyl
R¹¹ is hydrogen or a —C₁-C₄ alkyl.

55. The method of claim 54, wherein
R¹ and R¹⁰ represent a hydrogen, Br or —SO₃H;
R² and R⁹ represent a hydroxyl or methoxy;
R³ and R⁸ represent a hydroxyl or methoxy;
R⁴ and R⁷ represent a hydrogen, Br or —SO₃H;
R⁵ and R⁶ represent a hydroxyl or methoxy.

56. The method of claim 49, wherein said radiosensitizing agent is hexamethyl hypericin (HxMeHy) having the formula (I) wherein R¹, R⁴, R⁷ and R¹⁰ are H and R², R³, R⁵, R⁶, R⁸ and R⁹ are methoxy.

57. The method of claim 49, wherein said radiosensitizing agent is hypericin tetrasulfonic acid (HyTS) having the formula (I) wherein R¹, R⁴, R⁷ and R¹⁰ are —SO₃H and R², R³, R⁵, R⁶, R⁸ and R⁹ are hydroxyl.

58. The method of claim 49, wherein said radiosensitizing agent is tetrabromo hypericin (TBrHy) having the formula (I) wherein R¹, R⁴, R⁷ and R¹⁰ are Br and R², R³, R⁵, R⁶, R⁸ and R⁹ are hydroxyl.

59. The method of claim 49, wherein said cancer is a solid cancer.

60. The method of claim 59, wherein said cancer is selected from the group consisting of: melanomas, gliomas, breast cancer, colon cancer, pancreas cancer, prostate cancer, lung cancer, esophagus cancer, stomach cancer, liver cancer and head and neck cancer.

61. The method of claim 49, wherein said radiosensitizing agent being HxMeHy and said cancer is brain cancer and wherein said radiosensitizing agent being HyTS and said cancer is selected from the group consisting of breast cancer, pancreatic cancer and colon cancer and wherein said radiosensitizing agent being TBrHy and said cancer is selected from colon cancer, breast cancer, pancreatic cancer and brain cancer.

62. A method for sensitizing a specific cancer cell to radiation, the method comprising contacting cancer cells with an amount of an anthraquinone derivative of general formula (I), the amount being effective to specifically sensitize the cancer cells to radiation. 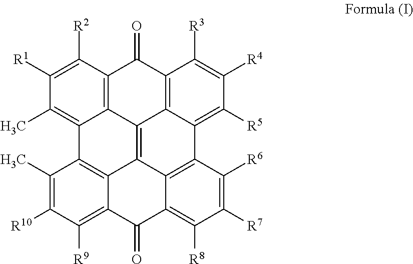
wherein
R¹ to R¹⁰ are substituents, each representing, independently, hydrogen, hydroxyl, —C₁-C₄ alkyl or —C₂-C₄ alkenyl, —C₁-C₄ alkoxy, —C₁-C₄ alkanoyl, —C₁-C₄alkanol, piperidinyl, halogen, —CX₃, —SO₃R¹¹, —R¹¹SO₂NH₂, —PO₃HR¹¹, —CO₂R¹¹, —COR¹¹; or any two adjacent substituents form together a cyclic or heterocyclic ring;
wherein
X representing a halogen; and
R¹¹ representing a hydrogen or a —C₁-C₄ alkyl;
or a salt, an ester or complex thereof, for use as a cancer specific radiosensitizing agent, provided that said anthraquinone derivative of formula (I) is not hypericin.

63. The method of claim 62, wherein
R¹ and R¹⁰ represent a hydrogen, halogen or an —SO₃R¹¹;
R² and R⁹ represent a hydroxyl or an alkoxyl;
R³ and R⁸ represent a hydroxyl or alkoxyl;
R⁴ and R⁷ represent a hydrogen, a halogen or an —SO₃R¹¹;
R⁵ and R⁶ represent a hydroxyl or an alkoxyl
R¹¹ is hydrogen or a —C₁-C₄ alkyl;

64. The method of claim 63, wherein
R¹ and R¹⁰ represent a hydrogen, Br or —SO₃H;
R² and R⁹ represent a hydroxyl or methoxy;
R³ and R⁸ represent a hydroxyl or methoxy;
R⁴ and R⁷ represent a hydrogen, Br or —SO₃H;
R⁵ and R⁶ represent a hydroxyl or methoxy.

65. The method of claim 64, wherein the anthraquinone derivative is selected from:
HxMeHy having the formula (I) wherein R¹, R⁴, R⁷ and R¹⁰ are H and R², R³, R⁵, R⁶, R⁸ and R⁹ are —OCH₃.
HyTS having the formula (I), wherein R¹, R⁴, R⁷ and R¹⁰ are —SO₃H and R², R³, R⁵, R⁶, R⁸ and R⁹ are OH.
TBrHy having the formula (I), wherein R¹, R⁴, R⁷ and R¹⁰ are Br and R², R³, R⁵, R⁶, R⁸ and R⁹ are OH.

66. The method of claim 62 comprising contacting brain cancer cells with an amount of HxMeHy, the amount being effective to specifically sensitize the brain cancer cells to radiation.

67. The method of claim 62 comprising contacting cancer cells selected from the group consisting of breast cancer, pancreatic cancer and colon cancer with an amount of HyTS, the amount being effective to specifically sensitize the cancer cells to radiation.

68. The method of claim 62 comprising contacting cancer cells selected from the group of colon cancer, breast cancer, pancreatic cancer and brain cancer to with an amount of TBrHy, the amount being effective to specifically sensitize the cancer cells to radiation.
